(19) United States
(12) Patent Application Publication
(10) Pub. No.: US 2021/0208070 A1
(43) Pub. Date: Jul. 8, 2021

(54) DETERMINATION OF ADULTERATED DIESEL FUEL USING AN ENVIRONMENTALLY SENSITIVE PHOTOLUMINESCENT MOLECULAR PROBE

(71) Applicant: Bundesrepublik Deutschland, vertreten durch die Bundesministerin für Wirtschaft und Energie, Berlin (DE)

(72) Inventors: Raúl Gotor, Berlin (DE); Jérémy Bell, Berlin (DE); Knut Rurack, Berlin (DE)

(73) Assignee: Bundesrepublik Deutschland, vertreten durch die Bundesministerin für Wirtschaft und Energie, Berlin (DE)

(21) Appl. No.: 16/650,418

(22) PCT Filed: Sep. 29, 2017
(86) PCT No.: PCT/EP2017/074885

§ 371 Date: Mar. 25, 2020

(51) Int. Cl. G01N 21/64; C10L 1/00; G01N 33/28

(52) U.S. Cl. CPC G01N 21/643; G01N 2021/6439; G01N 33/2882; C10L 1/003

(57) ABSTRACT

A method for detection of an adulterated diesel fuel in a sample is disclosed. The method includes contacting a sample with a molecular probe, the molecular probe having a photoluminescence which is environmentally sensitive; collecting the photoluminescence from the molecular probe; and determining whether the photoluminescence is indicative of adulterated diesel fuel. A test strip for the detection of adulterated diesel fuel in a sample is disclosed, comprising a molecular probe embedded in a substrate and/or immobilized to the substrate, the molecular probe having a photoluminescence which is environmentally sensitive to adulterated diesel fuel. The method and test strips are designed to be robust, portable, and within the capabilities of untrained personnel.

### FIELD AND BACKGROUND

The present invention relates to diesel fuel, particularly the detection of the adulteration of diesel fuel such as with kerosene. The present invention relates to colorimetric chemical analytical techniques for the detection of adulterated diesel fuel.

Diesel fuel adulteration can reduce engine performance, increase the chance for engine failure and contribute to environmental pollution. Adulteration of diesel fuel with kerosene can result in the emission of pollutants such as SOₓ derivatives, because kerosene can contain a high level of sulfur.

Simple reliable tests for diesel fuel adulteration are needed. In methods of mineral oil analysis, there has been substantial progress in the past decades, but most of the methods and techniques are complex, expensive, and/or unsuitable for on-site measurement. Diverse techniques such as density and evaporation measurements, distillation, measurement of ash content, the use of dye markers, infrared (IR) spectroscopy, fiber-optic techniques, and gas chromatography-flame ionization detection (GC-FID) are possible. However, robust, scalable, economic, simple-to-use, and portable methods are still lacking.

### SUMMARY

Against this background, disclosed herein is a method for detection of adulterated diesel fuel in a sample, making use of a molecular probe which has an environmentally sensitive photoluminescence. In some embodiments, the probe is immobilized to a test strip. Disclosed herein is a method and a test strip. Further configurations, details, and features of the present invention are also described herein.

Herein is disclosed a method for detection of an adulterated diesel fuel in a sample. The method includes contacting a sample with a molecular probe, the molecular probe having a photoluminescence which is environmentally sensitive. The photoluminescence from the molecular probe is collected. The method includes determining whether the photoluminescence is indicative of adulterated diesel fuel. The disclosed method provides a rapid, portable, and inexpensive analysis that does not require extensive training to perform.

According to a further embodiment, which can be combined with any other embodiment disclosed herein, the molecular probe is environmentally sensitive to viscosity and/or polarity. A molecular probe that is particularly sensitive to viscosity and/or polarity is advantageous because these properties of diesel fuel can be impacted by adulteration. Without being bound by theory, a method which uses a molecular probe which is sensitive to the viscosity of the environment is particularly contemplated.

According to a further embodiment, which can be combined with any other embodiment disclosed herein, the molecular probe has a twisted intramolecular charge transfer state, the twisted intramolecular charge transfer state inducing less photoluminescence than another state, such as a planar state. The twisted intramolecular charge transfer state may be variably accessible, such as being dependent on environmental conditions such as viscosity and/or polarity. A probe with a twisted intramolecular charge transfer state can be advantageous because such states can be variably accessible depending on the environment of the molecular probe, and/or such states can undergo environmentally sensitive processes. The environmental sensitivity of the molecular probe can affect the photoluminescence of the molecular probe, so that the photoluminescence can be used to determine if the sample is indicative of adulterated diesel fuel.

According to a further embodiment, which can be combined with any other embodiment disclosed herein, the molecular probe is a molecular rotor. A molecular probe which is a molecular rotor can be particularly environmentally sensitive, such as to viscosity of the sample. For example, a molecular rotor's rate of rotation or rate of transition from one configuration to another configuration may be particularly sensitive to the environment, such as the viscosity of the environment.

According to a further embodiment, which can be combined with any other embodiment disclosed herein, the molecular probe comprises a 4-nitrostilbene moiety, such as according to the formula

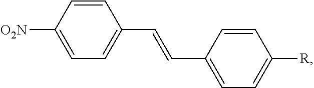

wherein R is selected from

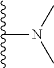 [SMILES: *N(C)C]

referred to as 4-DNS;

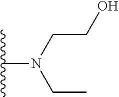 [SMILES: *N(CC)CCO]

referred to as 4-DNS-OH;

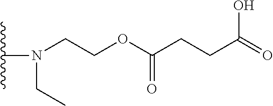 [SMILES: *N(CC)CCOC(=O)CCC(=O)O]

referred to as 4-DNS-COOH; and
a species immobilizing the molecular probe to a substrate, for example, covalently immobilizing the molecular probe. Using a 4-nitrostilbene moiety, such as those mentioned above, can be advantageous because they can provide an environmentally sensitive photoluminescence. The 4-nitrostilbene based species can be used for the detection of adulterated diesel fuel in a sample. According to a further embodiment, R includes a functional group resulting from the covalent immobilization of a molecular probe which includes a functional group for immobilizing the molecular probe, such as an alkoxyl, alkyl halide, primary amine, carboxylic acid, isothiocyanate, epoxide, azide, alkyne, phosphate or phosphoryl group, aldehyde, N-succinimdyl ester, or maleimide; and the immobilized molecular probe optionally includes a spacer group, such as for reducing the interaction of the substrate with the molecular probe, such as an interaction which sterically hinders the sample from contacting the immobilized molecular probe.

According to a further embodiment, which can be combined with any other embodiment disclosed herein, the molecular probe comprises 4-DNS-OH, which can be advantageous for detecting adulterated diesel fuel in a sample.

According to a further embodiment, which can be combined with any other embodiment disclosed herein, the molecular probe is embedded in a matrix on a substrate and/or immobilized on the substrate, such as adsorbed, ionically bonded, and/or covalently bonded; the substrate optionally being a test-strip or being on a test-strip.

A molecular probe that is embedded and/or immobilized to a substrate can provide a portable, stable, and easy to use form of the molecular probe. The adsorbed form is particularly easy to prepare since it does not require specific chemical linking of functional groups of the molecular probe with those of the substrate.

According to a further embodiment, which can be combined with any other embodiment disclosed herein, the substrate is selected from a group consisting of a cellulose, a nitrocellulose, a fabric, a glass fiber, an organic polymer, an inorganic fiber, and any combination thereof; the substrate optionally being a fiber and/or a paper. These substrates can be desirable for supporting an embedded/immobilized form of the molecular probe.

According to a further embodiment, which can be combined with any other embodiment disclosed herein, the sample is diesel fuel, optionally treated before contacting the sample with the molecular probe to substantially remove autofluorescent species such as polycyclic aromatic hydrocarbons and synthetic indicator dyes; wherein an optional treatment is with activated carbon. A method that works directly on diesel fuel is advantageous because it is simple for a user to do without extensive training. It can be advantageous to treat the sample to remove autofluorescent species in order to reduce background signals. Such treatment can make the method more sensitive to the detection of adulterated diesel fuel.

According to a further embodiment, which can be combined with any other embodiment disclosed herein, the method includes estimating a diesel content of the sample based on the photoluminescence. Such estimation can provide a user with more specific information to determine whether the diesel fuel is suitable for certain purposes or may require refining or disposal.

According to a further embodiment, which can be combined with any other embodiment disclosed herein, the sample is contacted to the molecular probe by dipping the substrate into the sample or dropping the sample onto the substrate or spraying the substrate with the sample. Dipping, dropping, or spraying can be advantageous in that they lead to adequate contact of the molecular probe and the sample, and can be performed by users without extensive training.

According to a further embodiment, which can be combined with any other embodiment disclosed herein, the method includes determining a signal, a brightness, a brightness ratio, a luminance, a photoluminescence quantum yield, a spectrum (such as a photoluminescence emission spectrum), and/or a photoluminescence kinetics such as a lifetime of the photoluminescence from the molecular probe in contact or after contact with the sample. A brightness ratio can be determined by collecting photoluminescence at two different wavelengths, for example. The use of different determinations, e.g. photoluminescent signal types and the like can provide greater sensitivity to the detection of adulterated diesel fuel.

According to a further embodiment, which can be combined with any other embodiment disclosed herein, a portable device collects the photoluminescence and determines whether the photoluminescence is indicative of adulterated diesel fuel; the portable device comprising optionally a lens and/or a fiberoptic for collecting the photoluminescence. The use of a portable device can be advantageous for allowing the method to be performed in remote areas. A lens and/or fiberoptic can be advantageous for conveniently allowing the photoluminescence to be collected.

According to a further embodiment, which can be combined with any other embodiment disclosed herein, the portable device is a smartphone or tablet, or any other mobile communication and computing device. These devices are particularly advantageous because users with little training can use them and they can operate in remote regions. It can be advantageous to have communication capabilities, because the device can conveniently transmit the data/results.

According to a further embodiment, which can be combined with any other embodiment disclosed herein, the method includes exciting the molecular probe with an ultraviolet or visible light source such as a camera flash, a LED, a laser, an incandescent light, and/or an ultraviolet source such as an ultraviolet LED. Exciting the molecular probe with such means is advantageous in that it provides a way to induce the photoluminescence.

According to a further embodiment, which can be combined with any other embodiment disclosed herein, the method includes comparing the photoluminescence to a calibration; such as comparing a signal, such as the luminescence, to a reference, such as stored data or a reference spot on test strip. The reference, such as stored data, can be tabular and/or a mathematical function, or the like. The reference can be remotely stored and available to the device through a communication link or can be locally stored, for example. It can be advantageous to have a reference such as a comparison so as to account for and possibly correct molecular probe photoluminescence variation that may not be directly caused by adulterated diesel fuel. The reference spot can take the form of a dot, line, and/or area, for example.

According to a further embodiment, which can be combined with any other embodiment disclosed herein, the molecular probe is covalently immobilized to a substrate and formed from a molecular probe which includes a functional group for covalently immobilizing the molecular probe to the substrate, the functional group being, for example, an alkoxyl, alkyl halide, primary amine, carboxylic acid, isothiocyanate, epoxide, azide, alkyne, phosphate or phosphoryl group, aldehyde, N-succinimdyl ester, or maleimide; the immobilized molecular probe optionally includes a spacer group, such as for reducing the interaction of the substrate with the molecular probe, such as an interaction which sterically hinders the sample from contacting the immobilized molecular probe.

Disclosed herein is a test strip for the detection of adulterated diesel fuel in a sample, including a molecular probe embedded in a substrate and/or immobilized to the substrate, the molecular probe having a photoluminescence which is environmentally sensitive to adulterated diesel fuel. The test strip can be advantageous for being portable, inexpensive, and easily used.

According to a further embodiment, which can be combined with any other embodiment disclosed herein, the molecular probe is environmentally sensitive to viscosity and/or polarity. A molecular probe that is particularly sensitive to viscosity and/or polarity is advantageous because these properties of diesel fuel can be impacted by adulteration.

According to a further embodiment, which can be combined with any other embodiment disclosed herein, the molecular probe has an accessible twisted intramolecular charge transfer state, the twisted intramolecular charge transfer state inducing less photoluminescence than another state, such as a planar state. A probe with a twisted intramolecular charge transfer state can be advantageous because such states can be variably accessible depending on the environment of the molecular probe, and/or such states can undergo environmentally sensitive processes. The environmental sensitivity of the molecular probe can affect the photoluminescence of the molecular probe, so that the photoluminescence can be used to determine if the sample is indicative of adulterated diesel fuel.

According to a further embodiment, which can be combined with any other embodiment disclosed herein, the molecular probe is a molecular rotor. A molecular probe which is a molecular rotor can be particularly environmentally sensitive, such as to viscosity of the sample.

According to a further embodiment, which can be combined with any other embodiment disclosed herein, the molecular probe comprises a 4-nitrostilbene moiety, such as according to the formula

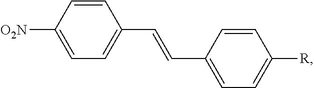

wherein R is selected from

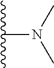 [SMILES: *N(C)C]

referred to as 4-DNS,

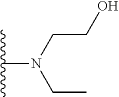 [SMILES: *N(CC)CCO]

referred to as 4-DNS-OH,

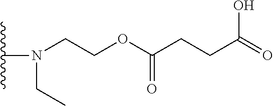 [SMILES: *N(CC)CCOC(=O)CCC(=O)O]

referred to as 4-DNS-COOH, and
a species immobilizing the molecular probe to a substrate, for example, covalently immobilizing the immobilized molecular probe. According to another embodiment, R includes a functional group resulting from the covalent immobilization of a molecular probe which includes a functional group for immobilizing the molecular probe, such as an alkoxyl, alkyl halide, primary amine, carboxylic acid, isothiocyanate, epoxide, azide, alkyne, phosphate or phosphoryl group, aldehyde, N-succinimdyl ester, or maleimide; and the immobilized molecular probe optionally includes a spacer group, such as for reducing the interaction of the substrate with the molecular probe, such as an interaction which hinders the sample from contacting the immobilized molecular probe.

Using a 4-nitrostilbene moiety, such as those mentioned above, can be advantageous because they can provide an environmentally sensitive photoluminescence. The 4-nitrostilbene based species can be used for the detection of adulterated diesel fuel in a sample.

According to a further embodiment, which can be combined with any other embodiment disclosed herein, the molecular probe comprises 4-DNS-OH, which can be advantageous for detecting adulterated diesel fuel in a sample.

According to a further embodiment, which can be combined with any other embodiment disclosed herein, the molecular probe is adsorbed, ionically bonded, and/or covalently bonded to the test strip.

It can be advantageous to embed/immobilize, or the like, the molecular probe to the substrate because it provides a portable, stable, and easy to use form of the molecular probe.

According to a further embodiment, which can be combined with any other embodiment disclosed herein, the substrate is selected from a cellulose, a nitrocellulose, a fabric, a glass fiber, an organic polymer, or an inorganic fiber; the substrate optionally being a fiber and/or paper. These substrates can be amenable for providing a support for an embedded/immobilized form of the molecular probe.

According to a further embodiment, which can be combined with any other embodiment disclosed herein, the test strip includes a reference photoluminescent species for comparison to the photoluminescence of the molecular probe; the reference photoluminescence species being optionally relatively environmentally insensitive. A reference can provide more information to determine whether the diesel fuel is adulterated, and may allow for correction of other effects that may influence the photoluminescence. Alternatively/additionally, multiple spots can allow for collection of more photoluminescence from the molecular probe and increase the confidence of the measurement.

According to a further embodiment, which can be combined with any other embodiment disclosed herein, the test strip includes multiple spots and/or lines of photoluminescent species, the photoluminescent species including the molecular probe. Multiple spots can provide for collection of more photoluminescence, possibly allowing for collecting photoluminescence from multiple samples, comparison of photoluminescence of the molecular probe to a reference, and/or acquisition of more data, and the like, for more robust sampling and more reliable results.

According to a further embodiment, which can be combined with any other embodiment disclosed herein, the test strip is covered entirely with molecular probe. This can be advantageous for providing a bright signal.

### BRIEF DESCRIPTION OF THE DRAWINGS

FIG. 1: A scheme of molecular rotors 4-DNS, 4-DNS-OH and 4-DNS-COOH.

FIG. 2: A. Emission of 4-DNS vs n-alkane chain length (Δ) and vs. kinematic viscosities (◯); B. Emission of 4-DNS-OH at 480 nm (Δ) and 543 nm (◯) vs diesel/kerosene blend viscosities; C. Emission of 4-DNS-OH in diesel (□), kerosene (Δ) and diesel/kerosene 1/1 v/v mixture (◯) vs temperature ([4-DNS-OH]=4 μM, λ_{exc}=430 nm); D. kinematic viscosity vs. diesel/kerosene mixtures.

FIG. 3: A. Emission of 4-DNS in pure alkanes from n-hexane (C6) to n-hexadecane (C16). B. Emission of 4-DNS in diesel upon increasing content of kerosene in the blend (0-100 vol %, λ_{exc}=430 nm).

FIG. 4: A. Absorption of 4-DNS-OH (in pentane), diesel, and Active Charcoal treated diesel (AC-Diesel), (Flask method). B. Emission of diesel and diesel after filtration with active charcoal filters. C. and D. Respective excitation-emission matrix for diesel and AC treated diesel with intensity scale.

FIG. 5: Images of test strips under UV lamp (365 nm) with adsorbed 4-DNS, 4-DNS-OH or 4-DNS-COOH (left to right; 10 μL of 1 mM toluene solutions), after 5 min elution in pure diesel

FIG. 6: Emission spectra of 4-DNS, 4-DNS-OH and 4-DNS-COOH adsorbed on standard cellulose paper before adding fuels or solvent.

FIG. 7 A. Emission of 4-DNS-OH test strips after dipping into various diesel/kerosene blends and plot of the integral fluorescence vs diesel content (λ_{exc}=430 nm). B. Images of 4-DNS-OH test strips after dipping into various diesel/kerosene blends, plus diesel alone without PAH removal and 4-DNS-OH. C, plot of the luminance vs. diesel content (λ_{exc}=365 nm);

FIG. 8: Normalized fluorescence intensities of 4-DNS-OH test strips after dipping in various liquids.

FIG. 9: A. Scheme and image of the smartphone case for test strip-based fluorescence analysis. (1) LED light source, (2) plastic diffuser, (3) 460 nm short pass filter, (4) test strip, (5) 550 nm band pass filter, (6) smartphone camera CCD. B. Screenshots of the application showing the strip's fluorescence once inside the measuring chamber and a menu with different options.

FIG. 10: Determined purity for different diesel/kerosene blends by means of GC-FID (Δ) and by molecular probe photoluminescence (◯) versus the real fraction of diesel.

### DETAILED DESCRIPTION

Herein, the terms “microenvironment” and “environment” may be used interchangeably in certain contexts, particularly when referring to the “environment” of a molecular probe. Herein the term PAH can refer to polycyclic aromatic hydrocarbons. Herein, the terms “dye,” and “indicator” may be used, in context, synonymously with “molecular probe” particularly when referring to a nonpolymeric photoluminescent species. A molecular probe which is grafted to a substrate, as described herein, is to be regarded as a molecular probe. Herein, “immobilized” may be used to describe a molecular probe which is associated with a substrate, such as physically adsorbed, chemically grafted, and the like. An “immobilized” molecular probe may be at least partially capable of eluting and/or desorbing when exposed to particular solvents. Herein a twisted intramolecular charge transfer state of a molecular probe can be “accessible” such as variable accessible. The rate of reconfiguration from a planar to a twisted intramolecular charge transfer state (and possibly vice versa) may be environmentally dependent, such as dependent on the local viscosity and/or polarity.

Herein, “active charcoal,” “active carbon,” “activated charcoal,” and “active charcoal” are used synonymously.

Adulteration of gasoline with kerosene, for example, can result in an annual loss of sales running into multiple millions. Such adulteration can harm the environment. It is possible to detect such adulterated diesel fuel, but many methods require expensive and complex equipment, experts, and/or laboratory environment. A simple method that can, for example, be used on the spot, e.g. at filling stations, is desired.

According to an embodiment described herein, a method for detection of an adulterated diesel fuel in a sample offers the possibility of an immediate, accurate measurement and analysis on the spot with a portable device such as a colorimeter and/or fluorimeter. For example, disclosed herein is a method including embedding/immobilizing molecular probes on a test strip which can facilitate analysis of a sample such that even untrained personnel can carry out the procedure.

Mineral oils can consist of linear and branched aliphatics, aromatic and non-aromatic cyclic hydrocarbons. The nonpolar nature of such materials and a lack of functional groups that can interact with probe or indicator molecules can make it difficult to rationally design chemical sensors. Surprisingly, we have found that alterations in global macroscopic properties such as polarity and/or viscosity can be more promising for analysis.

For example, molecular rotors can exhibit fluorescence properties that can depend on environmental viscosity and polarity. A rotor may have significantly different photophysical properties depending on the molecular configuration of the rotor. One configuration may be more photoluminescent than the other. For example, some molecular probes can contain an electron donating (D) and an electron accepting (A) group on opposite sides of a conjugated n system, which itself can include at least one single bond around which the two moieties D and A can rotate. Upon optical excitation, an intramolecular charge transfer process can occur which can be connected to a twisting of the D and the A unit against one another.

A molecular probe can have an accessible twisted intramolecular charge transfer state that can be variably accessible, such as depending on the environment. A more viscous environment may hinder the reconfiguration of a molecular probe in comparison to a less viscous environment. This can lead to a discernable difference of the photoluminescence of the molecular probe in liquids of varying viscosities.

For example, emission from a planar vibrationally relaxed Franck-Condon excited state of a molecular probe can be relatively strong, and emission from a twisted intramolecular charge transfer state (TICT) can be relatively weak. For example, given a fairly constant polarity environment around a molecular rotor, the molecular rotor's fluorescence can be influenced by the viscosity of its microenvironment. For example, if two liquids of interest are well miscible and possess comparable polarity yet distinctly different viscosity, the fluorescence intensity of a molecular probe (e.g. a molecular rotor) can be a function of the viscosity and can reflect the composition of the mixture of the two liquids.

Molecular rotors can be used as molecular probes which can be environmentally sensitive, such as sensitive to viscosity, particularly kinematic viscosity (ν). The addition of kerosene (ν=1.64 mm²·s⁻¹ at 27° C.) to diesel (ν=1.3-2.4, 1.9-4.1, 2.0-4.5 or 5.5-24.0 mm²·s⁻¹ at 40° C. for grades 1D, 2D, EN 590 or 4D) can reduce the mixture's kinematic viscosity (ν). The addition of kerosene to diesel can, for example, also result in a proportional fluorescence quenching of a photoluminescent molecular probe, particularly if the photoluminescence is environmentally sensitive.

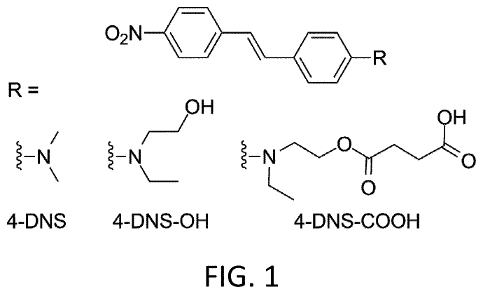

FIG. 1 shows a 4-dimethylamino-4-nitrostilbene (4-DNS) family of possible fluorescent molecular rotors, according to embodiments disclosed herein. Disclosed herein are molecular rotors 4-DNS, 4-DNS-OH and 4-DNS-COOH, according to exemplary embodiments, for a method of detection of diesel adulteration.

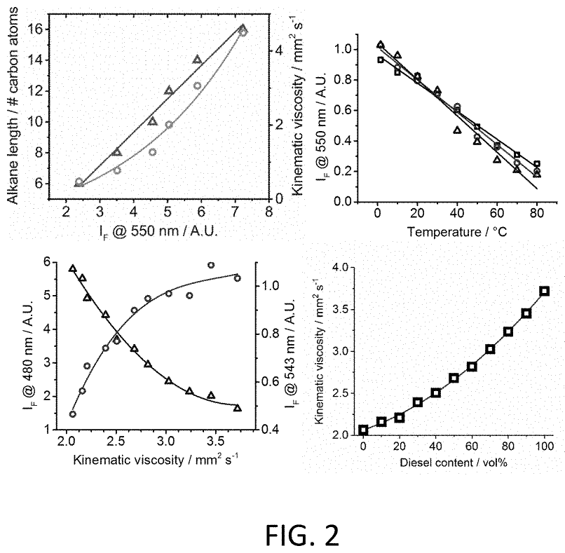

FIG. 2 illustrates, according to an embodiment, 4-DNS fluorescence properties upon variation of the kinematic viscosity in the viscosity range of 0.74-70.6 mm²·s⁻¹. This range reasonably matches the known viscosity values for diesel and kerosene. The spectroscopic properties of the molecular rotor 4-DNS in pure n-alkanes ranging from pentane (C₅H₁₂) to pentadecane (C₁₅H₃₂) can illustrate some principles of the method according to embodiments described herein.

FIG. 2(A) illustrates an increase of emission of 4-DNS with an increase in carbon chain length of solvent. The circles in FIG. 2(A) show the respective kinematic viscosities and emission of 4-DNS. The triangles show a corresponding relation between the emission of 4-DNS and viscosity.

FIG. 2(B) illustrates changes of fluorescence intensity at two wavelengths, 480 and 543 nm, with respect to kinematic viscosity of diesel-kerosene blends. It is possible that only very minor spectral changes may result in significant changes in the photoluminescence, e.g. photoluminescence intensity, at different wavelengths. Ratiometric methods are particularly contemplated. For example, the ratiometric parameter I(543)/I(480) may be used for determining the adulteration of diesel fuel, particularly with kerosene. Other ratios at other wavelengths may be even more sensitive. Other parameters and ratiometric parameters are also contemplated. The use of other parameters such as fluorescence quantum yield can also be utilized in determining whether the photoluminescence of the molecular probe is indicative of adulterated diesel fuel.

Without being bound by theory, the photoluminescence trends of 4-DNS that are observed in alkanes, e.g. as shown in FIG. 2(A), can also be compared to the photoluminescence trends (e.g. emission intensity of 4-DNS (c=4 μM) at 550 nm) upon changes of viscosity of various diesel/kerosene blends, as shown in FIG. 2(B).

Other factors, particularly temperature, may influence the photoluminescence of a molecular probe (e.g. 4-DNS-OH). FIG. 2(C) illustrates that temperature can influence the photoluminescence of 4-DNS derivatives, for example. FIG. 2(C) shows emission of 4-DNS-OH in diesel (squares), kerosene (triangles), and diesel-kerosene 1:1 v:v mixture (circles) versus temperature. It is possible to utilize references, calibration curves, and the like to correct for extraneous or other effects on the photoluminescence that are due to factors other than the adulteration of diesel fuel. A calibration and/or reference can be stored as data on the portable device and/or be available remotely such as by a communication link. A calibration and/or reference can take the form of a reference spot, line, or the like on a test strip.

The emission intensity of 4-DNS can also be influenced by temperature, both directly through molecular motions and indirectly through ν(T). This dependence can be accounted for by a correction and/or calibration. As illustrated in FIG. 2(C), a temperature increase can induce a concomitant decrease of the fluorescence intensity of 4-DNS irrespective of the liquid used, i.e., diesel, kerosene or a 1/1 mixture, and the dependence can be linear. Besides a slight gain or loss in sensitivity due to the absolute intensity changes, the influence of temperature can be corrected for.

FIG. 2(D) depicts the kinematic viscosity as influenced by the composition of a diesel/kerosene blend. FIG. 2(D) illustrates the concept, according to embodiments, that a molecular probe that is sensitive to viscosity can determine adulterated diesel fuel, such as diesel fuel which has be adulterated with kerosene.

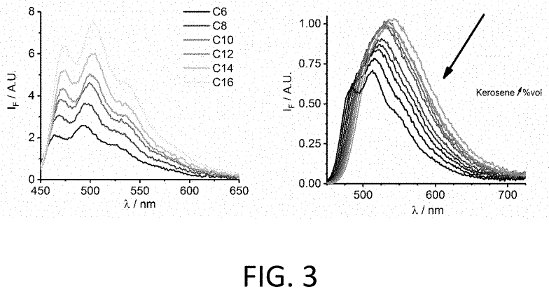

FIG. 3(A) illustrates emission of 4-DNS in pure alkanes from n-hexane to n-hexadecane. FIG. 3(B) illustrates emission of 4-DNS in diesel/kerosene mixtures of varying proportions. As kerosene content increases, the photoluminescence decreases. As an example, at T=24° C., the addition of kerosene to diesel can lead to fluorescence quenching down to 55% of the maximum recorded in pure diesel. The disclosed method for the detection of adulteration of diesel with kerosene can be reliable, having a standard deviation of 1.70%. Inspection of FIG. 3(B) indicates that several possible spectroscopic parameters can be used for determining the adulteration of diesel fuel with kerosene, such as photoluminescence intensity, and a ratiometric parameter (e.g. I(480)/I(550)).

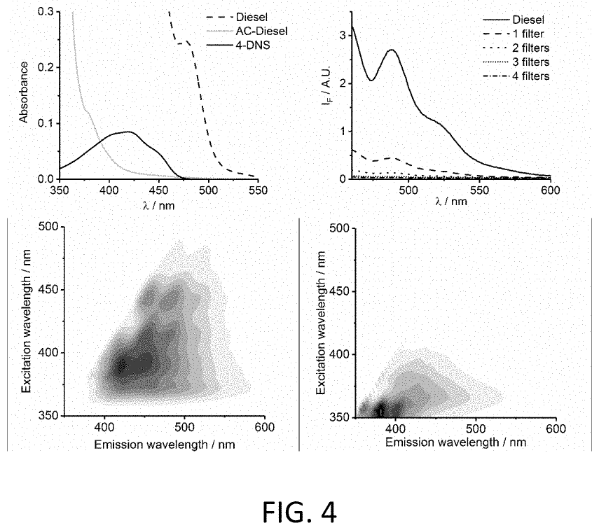

FIG. 4 illustrates, according to embodiments described herein, the absorption and emission of samples of diesel subjected to treatment for removal of autofluorescent species. Since diesel blends can be autofluorescent, for example due to the presence of fluorescent polycyclic aromatic hydrocarbons or even marker dyes, a treatment to substantially remove autofluorescent species can be part of the method, according to embodiments described herein. It may be advantageous to substantially remove autofluorescent species before contacting the sample with the molecular probe. For example, treatment of the sample with active charcoal can be done before the sample is contacted with the molecular probe. Such treatment can enhance reliability and minimize inconsistencies due to differences of diesel origin.

As examples, two types of pretreatments are disclosed. First a laboratory-based protocol in which 10 wt % of active charcoal is suspended in the sample, stirred for 1 h, centrifuged and filtered to remove the charcoal. A second method, for example, can be based on a stainless steel in-line filter holder (e.g. 47 mm, PALL) with active carbon paper filters (typically 4). For example, 5 mL of sample can be filtered, affording approximately 2 mL of PAH-free solution. In both cases, the PAHs can be successfully removed from the diesel and the spectroscopic window for the fluorescence measurement of 4-DNS is free of interferences.

FIG. 4(A) shows the absorbance of 4-DNS-OH in pentane, the absorbance of diesel, and the absorbance of diesel treated to substantially remove autofluorescent species. FIG. 4(A) shows that it is possible that an untreated diesel sample may include species that absorb up to about 500 nm. The molecular probe 4-DNS-OH absorbs up to about 470 nm. It can be desirable, in this case, using 4-DNS-OH as the molecular probe and this particular diesel fuel, to treat the sample to remove those species that absorb within the absorbance range of 4-DNS-OH, particularly if these species are significantly photoluminescent. Removing autofluorescent species can decrease unwanted background signal.

As a person skilled in the art appreciates, comparison of the absorbance spectrum of 4-DNS-OH of FIG. 4(A) and to the absorbance of the untreated and treated diesel spectra indicates that, after removal of autofluorescent species, 4-DNS-OH can be excited within its absorbance band in such a way to minimize absorption of the autofluorescent species. This can reduce unwanted background autofluorescence which may interfere with the photoluminescence of the molecular probe, particularly when exciting from about 420 nm to the long-wavelength-edge of the absorbance band of 4-DNS-OH.

A laser or LED may be used with a wavelength suitable for excitation of the molecular probe while reducing excitation of autofluorescent species so as to minimize background fluorescence. It is also possible to use an incandescent light source.

For the DNS molecular probe family disclosed herein, it may be desirable to use an excitation within a range of wavelengths such that the low wavelength is greater than 400, 410, 420, 430, 440, 450, 460, or 470 nm. Alternatively/additionally an LED or laser can be used, emitting at greater than 400, 410, 420, 420, 440, 450, 460 or 470 nm. For 4-DNS-OH, although the absorbance at the longer wavelengths of this range may be less than the peak absorbance wavelength, the longer wavelength excitation may avoid the excitation of residual autofluorescent species that may provide unwanted background.

FIG. 4(B) shows the emission of diesel fuel and diesel fuel after charcoal filtration. FIG. 4(B) illustrates that the removal of autofluorescent species can decrease and/or wavelength shift the background autofluorescence emission signal, e.g. blueshift.

FIGS. 4(C) and 4(D) show the respective excitation-emission matrix for diesel (FIG. 4(C)) and diesel that has been treated to remove autofluorescent species (FIG. 4(D)). As a person skilled in the art appreciates, comparison of the absorbance spectrum of 4-DNS-OH of FIG. 4(A) and the excitation-emission matrix of FIG. 4(D) indicates that after removal of autofluorescent species, 4-DNS-OH can be excited within its absorbance band such that the effect of unwanted background autofluorescence on the photoluminescence is minimized, particularly when exciting from about 400 nm to the long-wavelength-edge of the absorbance band of 4-DNS-OH. However, as a skilled person appreciates, the relative photoluminescence quantum yield of 4-DNS-OH compared to that of the autofluorescent species within the sample, and other factors, can also influence the extent of background interference.

### Immobilization

It is particularly desirable to provide for a method which can utilize a molecular probe that is embedded in a matrix on a substrate and/or immobilized on a substrate, such as a test-strip. The substrate can optionally be a test-strip. The sample can contact the molecular probe by dipping the test-strip into the sample, for example. Two approaches are mentioned, as examples: (a) simple adsorption of the molecular probe on paper strips and (b) covalent grafting of the molecular probe onto the paper, such as after specific functionalization of the molecular probe and/or substrate.

It is to be appreciated that the photoluminescence properties of the molecular probe can be different when it is immobilized to a substrate in comparison to the solvated form of the molecular probe. Therefore, it may be advisable to carry out tests to confirm that the immobilized form of the molecular probe functions adequately as an indicator as desired. In an embodiment, the choice of substrate, molecular probe, and immobilization means are selected so as to provide for an environmentally sensitive immobilized molecular probe. The immobilized molecular probe's environmental sensitivity may be sensitive to the viscosity of a sample applied to the substrate, for example, which may provide for a means to test for adulterated diesel fuel.

The fabrication of the test strips can be straightforward, particularly when the immobilization of the molecular probe is by adsorption.

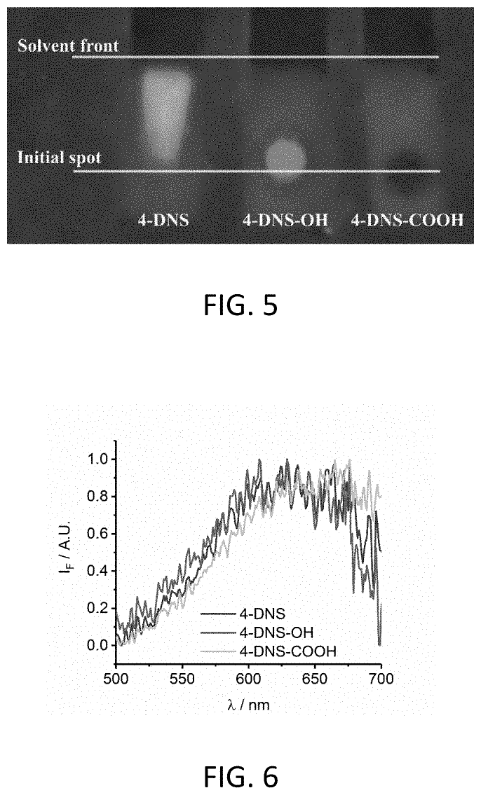

FIG. 5 shows a test strip image under UV excitation with adsorbed 4-DNS, 4-DNS-OH, and 4-DNS-COOH (left to right). The image is taken after elution in diesel. An elution of adsorbed 4-DNS was observed when dipping the strip into the liquid sample. Diesel can dissolve and elute 4-DNS. The more polar 4-DNS-OH and 4-DNS-COOH remained in the cellulose network. Without being bound by theory, this may be due to multiple hydrogen-bond interactions. Adsorbed 4-DNS-OH and 4-DNS-COOH can be more suitable as a molecular probe in terms of their resistance to elution. However, the suitability as a molecular probe for the determination of diesel fuel alteration also can depend on other factors. With the DNS family, although the fluorescence properties of the three molecular probes of FIG. 1 are similar in solution, their properties on cellulose paper can be different.

FIG. 6 illustrates, according to an embodiment, that the immobilized DNS based probes show a similar fluorescence at about 625 nm, without the influence of fuel or solvent in the environment. Without being bound by theory, the increasing polarity of the terminal functional group at the amino substituent (-Me<—OH<—COOH) can influence the spectroscopic response of the DNS based molecular probes. According to the data in TABLE 1, the similar fluorescence at about 625 nm suggests that when the DNS based molecular probes are adsorbed to cellulose fibers, they experience an environment comparable to alkyl ethers (e.g. di-n-butylether or 1,4-dioxane).

After wetting with fuel, the tests strips prepared with 4-DNS and 4-DNS-OH exhibited a hypsochromic shift and an enhancement of the emission, indicating effective solvation by the non-polar liquid (FIG. 5). Such effect was not observed for 4-DNS-COOH, which can be due to stronger interactions with the cellulose fibers which may prevent effective solvation by fuels. Moreover, the difference of the fluorescence intensity between a diesel and a kerosene environment, and therefore the sensitivity of the test, was also reduced the more polar the substituent was.

According to an embodiment that can be combined with any other embodiment described herein, a functional group (such as the functional group R as shown in FIG. 1) of the molecular probe can be varied according the resulting strength of the adsorption interaction with a substrate. A substrate and R group of the molecular probe can be selected to have favorable properties, particularly the resistance of the molecular probe to elution and maintenance of the environmental sensitivity of the photoluminescence of the molecular probe in the immobilized form.

It is understood that the resistance of the immobilized molecular probe to desorption from the substrate and the environmental sensitivity of the immobilized form of the molecular probe may be in tension. An R group of the molecular probe and a substrate can be selected so that the immobilized molecular probe resists being rapidly dissolved (particularly rapidly irreversibly desorbed from the substrate) in the sample, and the molecular probe remains environmentally sensitive to the sample. In this context, “rapidly” is intended to be understood as occurring such that it is difficult or impossible to obtain a photoluminescence signal from the immobilized molecular probe after/during contact with the sample.

According to an embodiment that can be combined with any other embodiment described herein, and with reference to FIG. 1, the R functional group can be selected from the group consisting of alkyl, alkoxy, halogen, alkyl halide, carboxyl, phosphate, and phosphoryl, and combinations thereof.

According to an embodiment that can be combined with any other embodiment described herein, the substrate can be selected from a cellulose, a nitrocellulose, a fabric, a glass fiber, an organic polymer, or an inorganic fiber; the substrate optionally being a fiber and/or paper.

According to an embodiment, which may be combined with any other embodiment described herein, the molecular probe is immobilized to the substrate so as to allow for a negligible amount of desorption upon exposure to the sample, such as an amount of desorption being adequate to show that the photoluminescence of the molecular probe is strongly influenced by the sample, particularly a liquid sample, rather than the substrate surface. Contacting the molecular probe with the sample may not quantitatively remove the probe from the substrate, but may allow for intermolecular interaction with the sample.

According to an embodiment, a molecular probe, is grafted onto a substrate that is suitable for maintaining the environmental sensitivity of the molecular probe. For the understanding of the invention, 4-DNS-COOH can be coupled to previously aminated Whatman 1 filter paper via standard NHS/DCC (N-Hydroxysuccinimide/Dicyclohexylcarbodiimide) coupling chemistry in dimethylformamide (DMF) (see Details of Exemplary Embodiments below). For 4-DNS-COOH adsorbed on paper, this material exhibited considerably weak emission, even in the presence of viscous substances. According to an embodiment, the molecular rotor 4-DNS-OH, adsorbed on substrates such as paper and cellulose, is suitable as a molecular probe for the detection of an adulterated diesel fuel in a sample. 4-DNS-OH combines a strong enough interaction with the cellulose to avoid elution, but also has an effective turn on of the fluorescence upon increasing the proportion of kerosene. As an example, the characterization of 4-DNS-OH paper strips yielded an amount of the molecular probe on the paper of 1.83±0.15 μmol_{dye}·g⁻¹ₚₐₚₑᵣ.

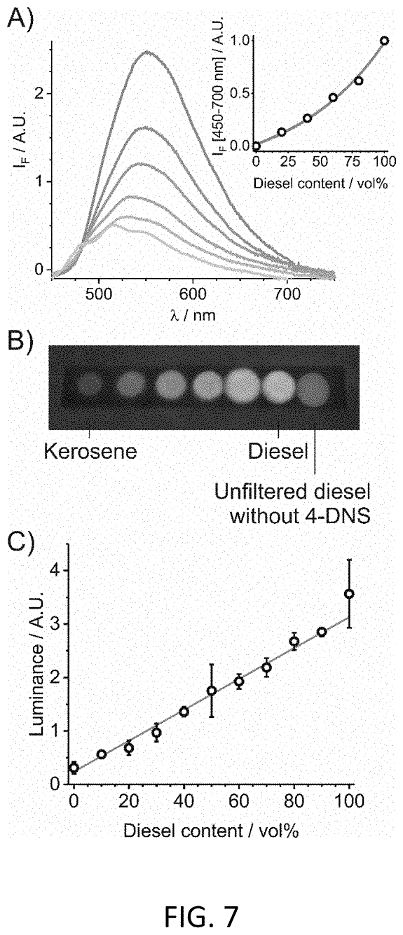

FIGS. 7(A), 7(B), and 7(C) illustrate, according to embodiments described herein, the response of 4-DNS-OH test strips toward various diesel/kerosene blends. The response was studied in parallel with a spectrometer and a digital camera. For digital camera analyses, the samples were placed over a white surface, and illuminated with a UV lamp at 365 nm. The camera (Canon Powershot S90) was placed at 150 mm over the strips, and its parameters were adjusted to fit the linear range of the CMOS and average out possible interferences from the 60 Hz of the UV-lamp (f 3.5, speed 1/10 s and IS01600). Images of the test strips with 4-DNS-OH and 10 μL of the different analyte mixtures were taken within 10 s after the addition of the sample to the substrate.

A fluorescence intensity decrease, a hypsochromic shift from 550 to 515 nm, and the appearance of a more structured band shape were observed upon increasing the kerosene content of the blend (FIG. 7(A). FIG. 7(A), inset, depicts the integral fluorescence of 4-DNS-OH test strips after dipping the test strip into various diesel-kerosene blends, excitation being at 430 nm, the collected photoluminescence in a band from 450-700 nm. According to embodiments described herein, the collected photoluminescence can be filtered by an appropriate high pass filter to remove excitation light.

The collected photoluminescence may be compared to a reference, to determine the diesel content, for example. The inset of FIG. 7(A) may be regarded as illustrative of reference data, which may take a tabular or functional form. According to embodiments described herein, collected photoluminescence from a molecular probe which has been in contact with a sample may be compared to the reference to determine whether the photoluminescence is indicative of adulterated diesel fuel and/or to estimate diesel content, and/or adulterant content (particularly kerosene).

FIG. 7(B) depicts, according to an embodiment described herein, an image of test strips prepared from 4-DNS-OH after dipping into various diesel/kerosene blends, and diesel with unremoved PAH. FIG. 7(C) illustrates the luminance vs. diesel content, with excitation at 365 nm. FIGS. 7(A) inset and 7(C) illustrate that the photoluminescence can be collected under various conditions, for example, the photoluminescence can be collected from excitation at different wavelengths. The determination of whether the photoluminescence is indicative of adulterated diesel fuel may utilize different kinds of photoluminescence data. The collected photoluminescence data is not limited to spectra, intensity, and luminance, but may also include lifetimes (e.g. bleach rates, photoluminescence lifetime, photoluminescence quantum yield). The embodiments shown in FIG. 7 are illustrative of the possibility of using intensity and/or luminescence, upon excitation at multiple wavelengths (e.g. visible and ultraviolet) and collection of photoluminescence in variable spectral ranges. For example, photoluminescence can be collected from an ultraviolet light excited molecular probe; and photoluminescence can be collected from a visible-light excited molecular probe. Such techniques may add to the reliability of the method.

The fluorescence intensity of 4-DNS-OH strips showed a linear correlation (r²=0.997) with diesel content for both spectrometer and camera, and a low standard deviation of 2.5% (FIGS. 7(A) and (B)).

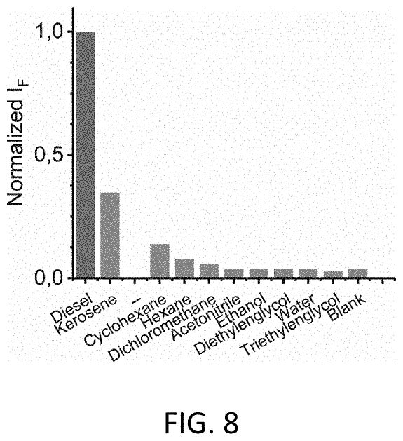

It can be possible that traces of polar compounds in diesel do not extensively promote non-radiative pathways, such as the extensive population of TICT or other charge transfer states. Such phenomena may be more pronounced in polar solvents, whether viscous or non-viscous: ethanol, water, acetonitrile, diethylene glycol or triethylene glycol (FIG. 8). FIG. 8 illustrates normalized fluorescence intensities of 4-DNS-OH test strips after dipping in possible interferents. Non-viscous and non-polar solvents (hexane, cyclohexane or dichloromethane) may produce weak responses when spotted on the test strips (<10%).

### Portable Devices

According to embodiments described herein, a portable device such as a smartphone, tablet, or mobile communication and computing device collects the photoluminescence and determines whether the photoluminescence is indicative of adulterated diesel fuel. The portable device includes, optionally, a lens and/or a fiberoptic for collecting the photoluminescence; a digital camera can be used.

We describe herein a system for detection of an adulterated diesel fuel in a sample. For fluorescence sensors, such as a test strip, the systems can include a dark chamber and a controlled excitation source, such as a camera flash, an LED, a laser, an incandescent light, and/or an ultraviolet source such as an ultraviolet LED, to yield accurate and reproducible results. Measurement systems using a smartphone or another mobile communication device, such as a tablet, can be suitable for on-site testing by unskilled personnel. There may be more than one excitation source, such as for collecting photoluminescence which is excited at two different wavelengths, such as in the UV and visible. The device may also include optical filters for filtering the excitation and/or collected photoluminescence.

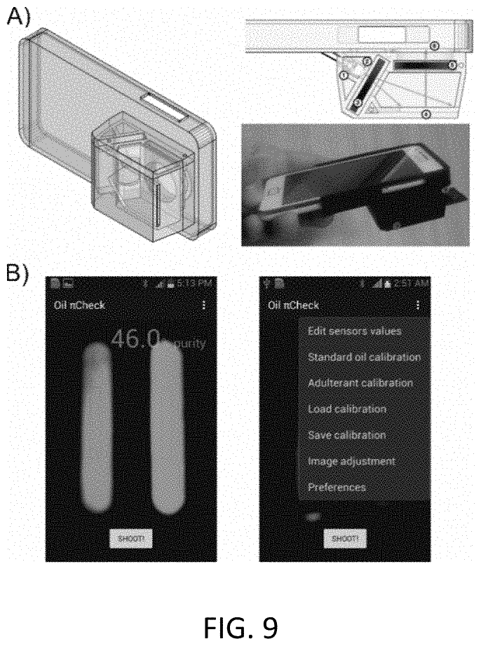

Herein a smartphone measurement system capable of analyzing the fluorescence response of 4-DNS-OH test strips is disclosed. For example, a system based on a Samsung Galaxy S2 was designed, integrating optical elements (FIG. 9A-B). Device operation and data analysis was carried out with a Java application for Android. The 3D printed smartphone case consisted of a black chamber (20×30×40 mm) with a standard LED at 460 nm as excitation source driven by the 20 mA DC current drawn from the smartphone battery via an USB on-the-go (OTG) connection. The excitation was diffused through a small polyethylene diffuser and filtered (Semrock FF01-492/SP) before illuminating the paper strips at an angle of 60°. Paper strips were placed in a holder and fluorescence was measured through a filter (Semrock, FF01-550/49) with the smartphone CCD camera. In the strip holder, beside the test strip, a paper strip coated with a fluorescent boron-dipyrromethene (BODIPY) dye (BDP), (see Details of Exemplary embodiment below) was used as reference material to correct the auto-exposure fluctuation of the camera.

Some smartphone models can be equipped with means to allow users and/or programmers to access or control the exposure and shutter speed of the camera. It can be advantageous to obtain suitable raw images from camera acquisition, e.g. images that do not suffer from auto-exposure compensation algorithms. Such algorithms, which may be integrated into a smartphone hardware or software, can be convenient for an end user as a hobby photographer, but may pose problems when using the smartphone for chemical analysis and chemometric techniques. For example, the lux amount received by the camera's detector such as CMOS or CCD can possibly be automatically tuned to match certain predefined lux criteria. Using such values instead of properly calibrated and corrected ones can lead to misleading and false results.

According to an embodiment, the method of determining diesel adulteration can include comparing the photoluminescence to a calibration; such as comparing a signal, such as the luminescence, to a reference. The reference may be stored data or a reference spot on test strip, for example.

According to an embodiment, implementation of a reference, such as a reference strip placed beside the test strip, to take into account the smartphone's auto-exposure compensation can be utilized. For example, after a photograph of the reference strip and the test strip after having been dipped into a sample has been taken, the software can average all the RGB values of the pixels in predefined spatial areas corresponding to the strips, which can then be converted to fluorescence intensities (see Details of Exemplary embodiment as indicated below). A two-point calibration procedure with two reference solutions (pure diesel and pure kerosene) can be done to obtain and store calibration files depending on the fuel, for example, because the various diesel grades can possess specific viscosities and therefore specific responses with the test strips.

For validation purposes, for example, analyses of various kerosene blends can be carried out in parallel, such as by following two different methodologies, one being a GC-FID. Following common guidelines for hydrocarbon analysis, a GC-FID standard method can be used for measurement. GC-FID can allow differentiation of diesel blends by calculating the peak area ratios C40/C10 which, according to test results, led to a linear relationship (r²=0.99) with a standard deviation of 6% for such mixtures. The resulting linear calibration curve can be used to validate the results obtained with a smartphone-chemometric tandem system. Such data can be stored and used as a calibration, for example.

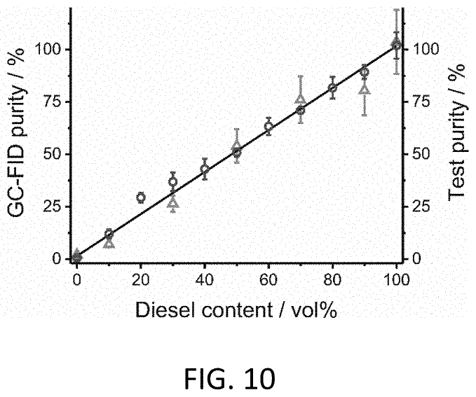

FIG. 10 depicts, according to an embodiment, a validation of the method. Diesel/kerosene mixtures of varying compositions were prepared. Circles depict the diesel content of the samples determined by the method using a molecular probe immobilized to a substrate and dipped into a liquid sample. Triangles depict the diesel content determined by GC-FID.

According to an embodiment, after appropriate selection of calibration and/or reference data, such as a calibration file from the software memory, the test strip which was previously loaded with the molecular rotor can be dipped into the sample. Next, excess of sample can be optionally removed by simple patting with a drying paper, and the test strip can be placed inside the smartphone case. The strip's fluorescence can then be checked on the smartphone's display and, after pressing a “measure” button, the photoluminescence can be collected. For example, the fluorescence intensity can be recorded. For example, the degree of adulteration can be calculated by the internal algorithm.

A linear response of the fluorescence intensity versus the diesel fraction may be obtained. Furthermore, the results obtained with the strip and those measured with a standard laboratory method (gas chromatography/flame ionization detector GC-FID) may agree well (FIG. 9). The good accuracy of 3% for the determination of the proportion of diesel was even better than that of the standard method and the uncertainties reported for similar sensors.

### Details of Exemplary Embodiments

The molecular probes 4-DNS and 4-DNS-OH and kerosene were used as commercially available. Reagents for synthetic procedures were obtained from commercial suppliers and used without further purification. Diesel was obtained from a HEM gas station at Berlin-Adlershof, Germany.

Air and moisture sensitive reactions were carried out using previously dried materials and N₂ atmosphere. Thin layer chromatography (TLC) analyses were performed over Merck Silica Gel 60 F₂₅₄ TLC. Reactions were monitored using a 254 nm handheld lamp. Column chromatography was carried out with Merck Silica gel 60 (0.040-0.063 mm). NMR spectra were recorded on a 600 MHz (151 MHz for ¹³C) Bruker AV 600 spectrometer at 300 K using residual protonated solvent signals as internal standard (¹H: δ (CDCl₃)=7.26 ppm and ¹³C: δ (CDCl₃)=77.16 ppm). Mass spectra were measured on a Micromass Q-TOF Ultima ESI.

UV-vis absorption spectra were recorded on a Specord 210-Plus spectrophotometer from Analytik Jena AG. Steady-state fluorescence measurements were carried out on a FluoroMax-4 spectrofluorometer from Horiba Jobin-Yvon Inc., New Jersey, using standard 10 mm path length quartz cells. All the solvents employed for the spectroscopic measurements were of UV spectroscopic grade (Aldrich). The absorbance and fluorescence spectra were recorded, ensuring that the temperature of the sample was always within 24±0.5° C. Each experiment was run in triplicate unless specified.

For solution analyses, 10 μL of 4-DNS solutions (1 mmol L⁻¹ in toluene) were added to 2.5 mL of the liquid analyte in a 10×10 mm quartz cuvette. After mild homogenization to avoid bubble formation and consequently possible fluorescence quenching mediated by triplet oxygen species, the emitted fluorescence after exciting the sample at 430 nm was registered.

Procedures

Synthesis of 4-DNS-COOH

50 mg (0.16 mmol) of 2-[Ethyl[4-[2-(4-nitrophenyl)ethenyl]phenyl]amino] ethanol, 2 mg (0.016 mmol) of 4-dimethylaminopyridine and 19.2 mg (0.192 mmol) of succinic anhydride were dissolved in 2 mL of dry dichloromethane under Ar atmosphere in a previously flame dried round bottom flask. Then, 11.6 μL (0.16 mmol) of Et₃N were added and the mixture left to react for 20 h indicated by quantitative consumption of the starting materials as observed by TLC. Next, 2 mL water were added to the mixture before acidification to pH 2 using acetic acid. The mixture was extracted two times with 10 mL dichloromethane, washed with NaCl (sat.) and dried with Na₂SO₄. Column chromatography using petroleum ether:EtOAc 1:9 as eluent yielded 49 mg (74%) of the desired product. ¹H NMR (600 MHz, DMSO-d₆) δ 8.17 (d, J=8.8 Hz, 2H), 7.75 (d, J=8.8 Hz, 2H), 7.49 (d, J=8.8 Hz, 2H), 7.41 (d, J=16.3 Hz, 1H), 7.10 (d, J=16.3 Hz, 1H), 6.75 (d, J=8.9 Hz, 2H), 4.18 (t, J=6.0 Hz, 2H), 3.58 (t, J=6.0 Hz, 2H), 3.43 (q, J=7.0 Hz, 2H), 2.50-2.45 (m, 4H), 1.10 (t, J=7.0 Hz, 3H), ¹³C NMR (151 MHz, DMSO) δ 173.36, 172.20, 147.99, 145.23, 145.13, 133.89, 128.76, 126.30, 124.03, 123.67, 120.95, 111.58, 61.52, 48.05, 44.57, 28.73, 28.63, 12.00. HR-MS (ESI+): m/z calculated for C₂₂H₂₅N₂O₆ [M+H]⁺: 413.1707, found: 413.1713.

Synthesis of Reference Dye BDP (Formula See Below)

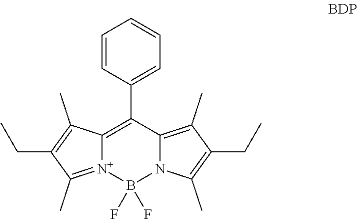

### 8-(phenyl)-1,3,5,7-tetramethyl-2,6-diethyl-4,4-difluoro-4-bora-3a,4a-diaza-s-indacene

The synthetic procedure was adopted from Coskun, A.; Akkaya, E. U., J. Am. Chem. Soc. 2005, 127, 10464-10465. The crude product was purified by column chromatography on silica using toluene as eluent to give compound BDP as bright reddish crystals (441 mg, 29%). ¹H-NMR (500 MHz, CDCl₃) δ 0.98 (t, 6H, J=7.6 Hz), 1.27 (s, 6H), 2.29 (q, 4H, J=7.6 Hz), 2.53 (s, 6H), 7.27-7.29 (m, 2H), 7.46-7.48 (m, 3H) ppm. HR-MS (ESI+): m/z calculated for C₂₃H₂₈BF₂N₂ [M+H]⁺: 381.2314, found: 381.2267.

Preparation of the Test Strips

Whatman filter paper 1 was cut into 30×5 mm strips, and around 50 of those strips (611 mg) were deposited in a sealable 5 mL vial together with a 4.5 mL of a 1 mM 4-DNS-OH toluene solution. The strips were agitated inside the vial with a vertical rotator for 20 min at 30 rpm. After that time, the toluene solution was poured out of the vial, and it was immediately filled with cyclohexane and rotated for 1 minute. This washing operation was repeated three times. After that, the strips could dry over a filter paper for 10 minutes. The measurement of the amount of adsorbed dye was calculated with the absorbance values after extraction of the dye with MeOH.

The photophysical properties of 4-DNS, 4-DNS-OH and 4-DNS-COOH are listed in table 1

TABLE 1
Photophysical properties of 4-DNS, 4-DNS—OH
and 4-DNS—COOH in various solvents

| | | λ_{abs}/ | λₑₘ/ | | Stokes |
|---|---|---|---|---|---|
| Compound | Solvent | nm | nm | φ_{F} | Shift/nm |
| 4-DNS | Toluene | 430 | 585 | 0.530 | 155 |
| 4-DNS | Kerosene | ^{b} | 502 | ^{a} | ^{b} |
| 4-DNS—OH | Cyclohexane | 421 | 510 | 0.183 | 89 |
| 4-DNS—OH | n-Hexane | 416 | 501 | 0.118 | 84 |
| 4-DNS—OH | Di-n-butylether | 432 | 577 | 0.096 | 145 |
| 4-DNS—OH | Toluene | 436 | 583 | 0.532 | 147 |
| 4-DNS—OH | 1,4-Dioxane | 438 | 661 | 0.107 | 222 |
| 4-DNS—OH | CHCl₃ | 436 | 738 | 0.027 | 295 |
| 4-DNS—OH | CH₂Cl₂ | 442 | 760 | 0.013 | 318 |
| 4-DNS—OH | Dimethylformamide | 455 | ^{c} | ^{c} | ^{c} |
| 4-DNS—OH | Acetonitrile | 442 | _{c} | ^{c} | _{c} |
| 4-DNS—OH | Ethanol | 436 | ^{c} | ^{c} | ^{c} |
| 4-DNS—OH | Triethyleneglycol | 457 | 730 | 0.009 | 273 |
| 4-DNS—OH | H₂O | 442 | ^{c} | ^{c} | ^{c} |
| 4-DNS—OH | Diesel | 429 | 542 | 0.434 | 113 |
| 4-DNS—OH | Kerosene | 423 | 512 | 0.247 | 89 |
| 4-DNS—OH | Gasoline | 430 | 602 | 0.102 | 172 |
| 4-DNS—OH | THF | 444 | 692 | 0.076 | 249 |
| 4-DNS—COOH | Kerosene | ^{b} | 508 | ^{a} | ^{b} |

^{a} Not calculated.
^{b} Not measured.
^{c} Too low/red-shifted to be measured.

Polycyclic Aromatic Hydrocarbons (PAH) Interferences

In order to check whether PAH interfere with the proposed test method, stock solutions of PAH-free diesel/kerosene blends with volume ratios of 10:0, 9:1, 8:2, 7:3, 6:4, 5:5, 4:6, 7:3, 8:2, 9:1 and 0:10 were prepared. These solutions were stored at 4° C. in sealed vials to avoid evaporation of hydrocarbons with higher vapor pressure.

Molecular Rotor and Viscosities of Various Diesel/Kerosene Blends

To ensure correct results, the viscosities of diesel/kerosene blends were measured according to ASTM standard D445 using a commercial calibrated Cannon-Fenske viscometer type 75 with constants of 0.00818 and 0.00815 respectively at 40° C. and 100° C. (FIG. 2(D)).

The product of the extrapolated constant to our working temperature (0.00802959 for 24° C.) and the efflux time yielded the kinematic viscosities of the blends which agreed with the theoretical viscosities obtained using Arrhenius equation. As expected, an increase of the diesel proportion in the blend, resulted in a non-linear (second order polynomial) increase of the kinematic viscosity.

Picture Analyses

Posterior analysis of the pictures was done with a custom image analysis software written in Processing 3. The software averaged the RGB values inside a selected circular area corresponding to the area where the oil blend was placed. The average was then transformed to the CIE 1931 color space, utilizing the standardized linear transformation stated by the CIE (Fairman, H. S.; Brill, M. H.; Hemmendinger, H., Color Res Appl 1997, 22, 11-23) and with a gamma correction of a 2.2 factor according to formula 1.

$$Formula 1. CIE standard linear transformation . \\ [ \begin{matrix}\overline{X} \\ \overline{Y} \\ \overline{Z}\end{matrix} ] = [ \begin{matrix}0.412453 & 0.357580 & 0.180423 \\ 0.212671 & 0.715160 & 0.072169 \\ 0.019334 & 0.119193 & 0.950227\end{matrix} ] \cdot [ \begin{matrix}\overline{R} \\ \overline{B} \\ \overline{G}\end{matrix} ]$$

The Y̅ parameter of this color space represents the luminance intensity of a given color, which was directly proportional to the fluorescence intensity of 4-DNS-OH adsorbed on paper. Each measurement of fluorescence made with the system was corrected afterwards using the stored calibration values for each diesel grade and kerosene according to formula 2:

$$Formula 2. Correction factors for the test \text{-} strips analyses$$

$${ON}_{f} = \frac{{I}_{s}^{d}}{{I}_{c}^{d}} {OFF}_{f} = \frac{{I}_{s}^{k}}{{I}_{c}^{k}}$$

where ON_{f} and OFF_{f} were the correction factors for the on and off states, Iₛ^{d} and Iₛ^{k} were the test strip intensities in pure diesel and pure kerosene, and I_{c}^{d} and I_{c}^{k} were the stored calibration intensities also for diesel and kerosene, respectively.

A second strip coated with the reference dye BDP in a defined concentration, affording therefore a constant fluorescence emission, was placed beside the sample strip. This constant reference was then coupled to the correction factors to correct the exposure fluctuation according to formula 3.

$$Formula 3. Exposure fluctuation correction$$

$${X}_{d} = \frac{{I}_{s} - ( {I}_{r}^{s} \times {OFF}_{f} )}{( {I}_{r}^{s} \times {ON}_{f} ) - ( {I}_{r}^{s} \times {OFF}_{f} )}$$

where X_{d} was the diesel fraction in the test sample and Iₛ and Iᵣ^{s} were respectively the intensities of test strip after dipping in the fuel blend and of the reference strip, respectively. The result was expressed as a fraction of the pure diesel and kerosene references.

Thus, a chemical system based on 4-DNS, a fluorescent molecular rotor sensitive to viscosity is proposed to obtain simple and efficient test strips for the detection of diesel fuel adulteration. The range of kinematic viscosities measured for diesel and its different blends with kerosene matched perfectly the system's response range, allowing the detection of small aliquots of kerosene. A derivative of the molecular rotor that can be adsorbed sterically in cellulose fiber networks without leaching, 4-DNS-OH, was then coated on paper to provide test strips stable upon dipping into fuel mixtures and giving a linear fluorescence response with increasing concentrations of kerosene. Finally, a smartphone case integrating an LED and a strip support was designed as well as an application to read, analyze and interpret the fluorescence signal. This complete and embedded handheld analysis system was compared to a standard method based on GC-FID for validation. The results obtained with both approaches agreed well, yielding linear responses and low limits of detection down to 7% of kerosene in diesel for the newly developed system. Such cost-effective, precise and rapid tests are a powerful forensic tool for consumers or unskilled personnel of investigative authorities, uncovering frauds.

The present invention has been explained with reference to various illustrative embodiments and examples. These embodiments and examples are not intended to restrict the scope of the invention, which is defined by the claims and their equivalents. As is apparent to one skilled in the art, the embodiments described herein can be implemented in various ways without departing from the scope of what is invented. Various features, aspects, and functions described in the embodiments can be combined with other embodiments.

1-29. (canceled)

30. A method for detection of an adulterated diesel fuel in a sample, the method comprising:
contacting a sample with a molecular probe, the molecular probe having a photoluminescence which is environmentally sensitive;
collecting the photoluminescence from the molecular probe;
determining whether the photoluminescence is indicative of adulterated diesel fuel.

31. The method of claim 30, wherein the molecular probe is environmentally sensitive to viscosity and/or polarity.

32. The method of claim 30, wherein the molecular probe has a twisted intramolecular charge transfer state, the twisted intramolecular charge transfer state inducing less photoluminescence than another state.

33. The method of any claim 30 the molecular probe is a molecular rotor.

34. The method of claim 30, wherein the molecular probe comprises a 4-nitrostilbene moiety, according to the formula 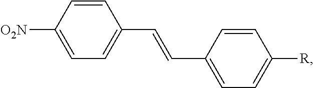
wherein R is selected from 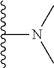 [SMILES: *N(C)C]
referred to as 4-DNS, 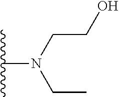 [SMILES: *N(CC)CCO]
referred to as 4-DNS-OH, 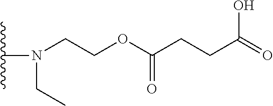 [SMILES: *N(CC)CCOC(=O)CCC(=O)O]
referred to as 4-DNS-COOH, and
a species immobilizing the molecular probe to a substrate.

35. The method according to claim 34, wherein
R includes a functional group resulting from the covalent immobilization of a molecular probe which includes a functional group for immobilizing the molecular probe, and the immobilized molecular probe includes a spacer group for reducing the interaction of the substrate with the molecular probe.

36. The method according to claim 30, wherein the molecular probe comprises 4-DNS-OH.

37. The method of claim 30, wherein the molecular probe is embedded in a matrix on a substrate and/or immobilized on the substrate; the substrate being a test-strip or being on a test-strip; and wherein the substrate is selected from the group consisting of a cellulose, a nitrocellulose, a fabric, a glass fiber, an organic polymer, an inorganic fiber, and any combination thereof; the substrate being a fiber and/or a paper.

38. The method of claim 30, wherein the sample is diesel fuel, treated before contacting the sample with the molecular probe to substantially remove autofluorescent species wherein the treatment is with activated carbon; and further comprising estimating a diesel content of the sample based on the photoluminescence.

39. The method of claim 30, wherein the sample is contacted to the molecular probe by dipping the substrate into the sample or dropping the sample onto the substrate or spraying the substrate with the sample.

40. The method of claim 30, further comprising determining a signal, a brightness, a brightness ratio, a luminance, a photoluminescence quantum yield, a spectrum, and/or a photoluminescence kinetics from the molecular probe in contact or after contact with the sample.

41. The method of claim 30, wherein a portable device collects the photoluminescence and determines whether the photoluminescence is indicative of adulterated diesel fuel; the portable device comprising a lens and/or a fiberoptic for collecting the photoluminescence, wherein the portable device is a smartphone or tablet, or any other mobile communication and computing device.

42. The method of claim 30, further comprising exciting the molecular probe with an ultraviolet or visible light source and/or an ultraviolet source.

43. The method of claim 30, further comprising comparing the photoluminescence to a calibration.

44. The method of claim 30, wherein the molecular probe is covalently immobilized to a substrate and formed from a molecular probe which includes a functional group for covalently immobilizing the molecular probe to the substrate, the immobilized molecular probe includes a spacer group for reducing the interaction of the substrate with the molecular probe.

45. A test strip for the detection of adulterated diesel fuel in a sample, comprising a molecular probe embedded in a substrate and/or immobilized to the substrate, the molecular probe having a photoluminescence which is environmentally sensitive to adulterated diesel fuel.

46. The test strip of claim 45, wherein the molecular probe is environmentally sensitive to viscosity and/or polarity.

47. The test strip of claim 45, wherein the molecular probe is a molecular rotor.

48. The test strip of claim 45, further comprising a reference photoluminescent species for comparison to the photoluminescence of the molecular probe; the reference photoluminescence species being relatively environmentally insensitive.

49. The test strip according to claim 45, wherein the test strip comprises multiple spots and/or lines of photoluminescent species, the photoluminescent species including the molecular probe.
